(19) United States
(12) Patent Application Publication
(10) Pub. No.: US 2006/0235289 A1
(43) Pub. Date: Oct. 19, 2006

(54) Pacemaker lead with motion sensor

(76) Inventors: Willem Wesselink, Doesburg (NL); Henricus W.M. De Bruyn, Arnhem (NL); Mattias Rouw, Arnhem (NL)

Correspondence Address: MEDTRONIC, INC., 710 MEDTRONIC PARK, MINNEAPOLIS, MN, 55432-9924, US

(21) Appl. No.: 11/109,391

(22) Filed: Apr. 19, 2005

(51) Int. Cl. A61B 5/05

(52) U.S. Cl. 600/407

(57) ABSTRACT

A system and method are provided for monitoring cardiac wall motion. A cardiac lead is provided with a motion sensor that is responsive to an excitation signal. The motion sensor signal induced by the excitation signal varies in time due to motion caused by myocardial wall motion. The time-varying motion sensor signal is obtained by sensing circuitry and provided to a processor for use in computing a wall motion parameter useful in assessing ventricular function. The processor provides wall motion parameter output for display or storage. Pacing parameters may be optimized according to wall motion parameter measurements obtained during iterative procedures using the lead-mounted motion sensor.

### FIELD OF THE INVENTION

The present invention relates generally to implantable cardiac leads and in particular to a cardiac lead having a motion sensor for detecting heart wall motion.

### BACKGROUND OF THE INVENTION

In some cardiac stimulation therapies, such as dual chamber pacing, cardiac resynchronization therapy, and extra systolic stimulation, it is desirable to optimize pacing parameters for achieving improved cardiac hemodynamic function. One parameter used to gauge cardiac function, particularly ventricular function, is the peak endocardial acceleration of the ventricular wall. In past practice, accelerometers have been included in cardiac leads for measuring endocardial acceleration for use in evaluating heart function on a chronic basis.

Pacing therapies, especially those delivered for treating heart failure, are preferably optimized to achieve a therapeutically beneficial improvement in heart function. A clinician will program pacing parameters in an implantable cardiac stimulation device at the time of implant and during patient follow-up visits. Pacing parameters, such as the timing between pulses delivered to different heart chambers, e.g., the atrial-ventricular delay (AV delay) or ventricular-ventricular delay (VV delay), can be optimized using some type of hemodynamic assessment, typically using ultrasound for measuring ejection fraction, stroke volume or wall displacement. Such optimization methods can be time consuming and require both an ultrasound technician and a clinician or other trained personnel to program the pacing parameters at various settings. It is desirable to provide a system and associated method that allows pacing optimization procedures to be performed in an efficient and reliable manner.

### BRIEF SUMMARY OF THE INVENTION

The present invention provides an implantable cardiac lead having a motion sensor at or near its distal end and an associated system and method for tracking myocardial wall motion. The cardiac lead may be a transvenous endocardial, coronary sinus lead or epicardial lead and is provided with a motion sensor located at or near the distal lead end. When the lead is deployed the motion sensor is positioned relative to a cardiac wall segment of interest such that wall segment motion is imparted onto the motion sensor. The motion sensor responds to an external excitation signal by producing a time-varying signal that is correlated to the motion of the myocardial wall segment during a cardiac cycle.

One aspect of the invention, in one embodiment, is a cardiac lead having a motion sensor provided as a receiver coil. The receiver coil is coupled to a sensing channel to sense motion in at least one dimension. The receiver coil is exposed to an externally applied electromagnetic field directed through the patient. The sensing channel senses the signal induced in the receiver coil. The sensed signal varies over the cardiac cycle due to myocardial wall motion and is used to compute a measurement of myocardial wall motion. In some embodiments, two or three orthogonally applied electromagnetic fields are directed through the patient to allow computation of a myocardial wall motion measurement in two or three dimensions, respectively.

In another embodiment, the motion sensor is embodied as an electrode used to measure a voltage signal in response to an externally applied low power RF signal. The induced voltage signal will vary over the cardiac cycle due to myocardial wall motion and can be used to compute a measurement of myocardial wall motion.

In some embodiments, the motion sensor signal may be calibrated to allow computation of actual wall excursion during a cardiac cycle. In a calibration procedure, a second motion sensor located on the cardiac lead at a known distance from the first motion sensor is used to calibrate the induced sensor signal.

In other embodiments the motion sensor is provided as an electronic circuit that resonates in response to an excitation signal transmitted through the patient. The motion sensor response to the excitation signal is received by a receiver antenna. The resonance of the motion sensor is sampled at a frequency high enough to contain cardiac wall motion information.

Another aspect of the invention, in one embodiment, is a system including an excitation signal module for transmitting a signal through a patient, a motion sensor located at or near the distal end of a cardiac lead that is responsive to the excitation signal; a sensing circuit for receiving a signal produced by the response of the motion sensor to the excitation signal; and a processor for computing a wall motion parameter from the received signal.

Another aspect of the invention, in one embodiment, is a method for measuring myocardial wall motion and using wall motion measurements or parameters derived there from for pacing parameter optimization procedures. In one embodiment, the derivative of the wall motion signal is used for determining a surrogate measure of the peak endocardial wall acceleration. Pacing parameter values such as AV delay and VV delay can be optimized based on peak endocardial wall acceleration or other wall motion related measurements as an indirect measure of contractility.

Another aspect of the present invention, in one embodiment, is a computer-readable medium for storing a set of instructions which, when implemented in a medical device system, cause the system to sense a signal produced by a motion sensor in response to an externally applied excitation signal, and compute a cardiac wall motion measurement from the sensed signal.

### BRIEF DESCRIPTION OF THE DRAWINGS

FIG. 1 is a simplified block diagram providing an overview of a cardiac wall motion sensing system provided in accordance with the invention.

FIG. 2 is a schematic diagram of a myocardial wall motion sensing system in accordance with one embodiment of the present invention.

FIG. 3 is a block diagram of typical functional components included in an implantable cardiac stimulation device, such as pacemaker 10 shown in FIG. 1.

FIG. 4A shows an electrocardiogram and a respiration signal.

FIG. 4B shows a signal sensed from a motion sensor.

FIG. 4C shows a cardiac wall motion sensor obtained after filtering the signal of FIG. 4B to remove respiratory motion.

FIG. 5 is a schematic diagram of a wall motion monitoring system according to an alternative embodiment of the present invention.

FIG. 6 is a flow chart summarizing steps included in a method for optimizing pacing parameters using a wall motion signal in accordance with the invention.

### DETAILED DESCRIPTION

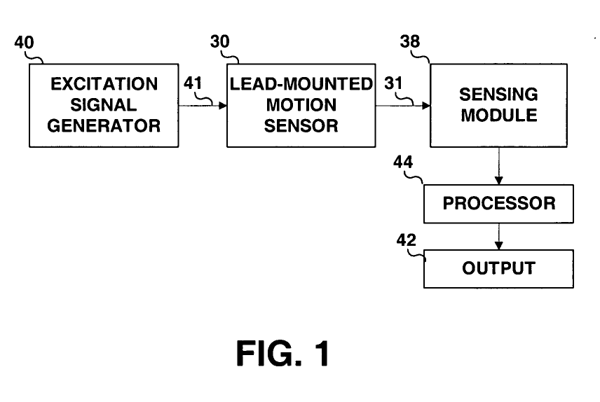

FIG. 1 is a simplified block diagram providing an overview of a cardiac wall motion sensing system provided in accordance with the invention. Generally, the system includes a lead mounted motion sensor 30 that is responsive to an excitation signal produced by excitation signal generator 40. As will be described herein, the system may be embodied as a magnetic or electromagnetic system wherein excitation signal generator 40 produces an (electro)magnetic field that induces a current in motion sensor 30, which is embodied as a receiver coil mounted at or near the distal end of a cardiac lead. The system may alternatively be an electronic system wherein excitation signal generator 40 produces radio-frequency (RF) signal that causes motion sensor 30, which can be embodied as an electronic circuit with an antenna or resonance strip, to resonate. Alternatively motion sensor 30 can be embodied as an electrode used for measuring an induced voltage signal in response to an externally applied, low power RF signal.

The response 31 of the motion sensor 30 to the excitation signal 41 is received by a sensing module 38. Sensing module 38 may be electrically coupled to motion sensor 30 to receive an induced current in an (E)M system or a voltage signal in an RF system. As such, sensing module 38 may be included in an implantable medical device used in conjunction with the lead carrying motion sensor 30. Sensing module 38 alternatively includes one or more antennas for receiving a resonating signal from motion sensor 30 in an RF electronic system. In such systems, sensing module 38 may be included in an external device and receive motion sensor signal 31 in a wireless manner. The sensing module 38 provides the motion sensor signal to processor 44 for computing a wall motion parameter from the sensed signal. The processor 44 generates wall motion data for receipt by output module 42 for presentation to a user, for example on a display or for storage by an electronic storage medium. A display of wall motion data may include an image of anatomical features. Processor 44 may also provide data for transmission to a clinician, medical center, central database or expert patient management center in which case output module 42 includes a wired or wireless networked communication interface for transferring data to a designated recipient or location.

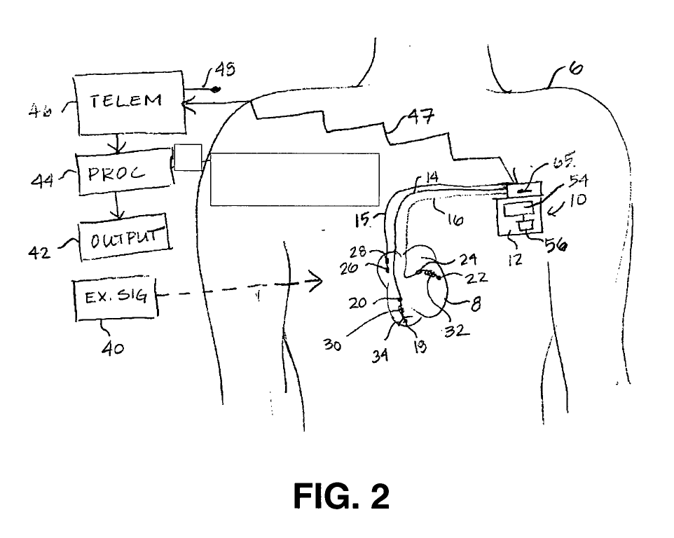

FIG. 2 is a schematic diagram of a myocardial wall motion sensing system in accordance with one embodiment of the present invention. An implantable cardiac stimulation device 10 is shown coupled to a set of cardiac leads 14 and 16 used for positioning electrodes and other physiological sensors relative to a patient's heart 8. Device 10 may be configured to integrate both monitoring and therapy features, as will be described below, and may include pacing, cardioversion and defibrillation therapies. In one embodiment, device 10 is embodied as a pacemaker having multi-chamber pacing capabilities. For the sake of illustration, cardiac stimulation device 10 is referred to hereafter as “pacemaker” 10 though it is recognized that the invention is not limited to implementation in a pacemaker but could also be implemented in a variety of implantable cardiac monitoring and/or stimulation devices.

The wall motion monitoring methods provided by the present invention can be used for optimizing pacing parameters used for controlling the delivery of a cardiac stimulation therapy such as, for example, dual-chamber pacing, cardiac resynchronization therapy, or extra-systolic stimulation. The wall motion monitoring methods may alternatively be used for monitoring purposes for assessing heart function. Pacemaker 10 collects and processes data from one or more sensors for determining physiological events and conditions and when cardiac stimulation therapies are required. In accordance with the present invention, pacemaker 10 receives a motion sensor signal for use in monitoring wall motion as will be fully described below.

Pacemaker 10 is provided with a hermetically-sealed housing 12 that encloses a processor 54, a digital memory 56, and other components as appropriate to produce the desired functionalities of pacemaker 10. Processor 54 may be implemented with any type of microprocessor, digital signal processor, application specific integrated circuit (ASIC), field programmable gate array (FPGA) or other integrated or discrete logic circuitry programmed or otherwise configured to provide functionality as described herein. Processor 54 executes instructions stored in digital memory 56 to provide functionality as described below. Instructions provided to processor 54 may be executed in any manner, using any data structures, architecture, programming language and/or other techniques. Digital memory 56 is any storage medium capable of maintaining digital data and instructions provided to processor 54 such as a static or dynamic random access memory (RAM), or any other electronic, magnetic, optical or other storage medium.

As further shown in FIG. 1, pacemaker 10 receives one or more cardiac leads for connection to circuitry enclosed within the housing 12. In the example shown, pacemaker 10 receives endocardial leads 14 and 15 and coronary sinus lead 16, although the particular cardiac leads used can vary from embodiment to embodiment. In addition, the housing 12 of pacemaker 10 may function as an electrode and be used for sensing EGM signals or used in combination with any lead-based electrode for delivering cardiac stimulation pulses.

Leads 14, 15 and 16 include pacing and sensing electrodes 18 and 20, 26 and 28, and 22 and 24, respectively, and may include high voltage coil electrodes (not shown) in the event pacemaker 10 is configured to provide cardioversion and/or defibrillation therapies. Lead 14 is shown as a transvenous endocardial lead used for deploying electrodes 18 and 20 in the right ventricle of the heart 8 for sensing right ventricular signals and delivering stimulation pulses in the right ventricle.

Endocardial lead 15 includes electrodes 26 and 28 for positioning in the right atrium for sensing right atrial signals and delivering right atrial stimulation pulses.

Lead 16 is shown as a coronary sinus lead used for deploying electrodes 22 and 24 in a cardiac vein over the left ventricle for sensing left ventricular signals and delivering stimulation pulses to the left ventricle. Coronary sinus lead 16 may include left atrial pacing and sensing electrodes in some embodiments. Electrodes carried by leads 14, 15 and 16 and housing 12 are used to deliver pacing stimuli in a coordinated fashion to provide dual-chamber pacing, biventricular pacing, cardiac resynchronization, extra systolic stimulation therapy or other benefits.

Pacemaker 10 may obtain other physiological signals, such as blood pressure signals, blood oxygen saturation signals, acoustical signals, activity sensor signals, or other physiological signals used in monitoring patient 6 and determining when a cardiac stimulation therapy is needed. Pacemaker 10 may receive physiological signals from sensors deployed on leads 14 or 16 or other auxiliary cardiac or subcutaneous leads or included on or in pacemaker housing 12.

In accordance with the present invention, one or more leads coupled to pacemaker 10 are provided with a motion sensor at or near the distal end of the lead. The end of a lead positioned in the heart 8 is referred to herein as the “distal end”, and the “proximal end” of the lead is the end coupled to pacemaker 10. In the example embodiment shown in FIG. 1, endocardial lead 14 is provided with a motion sensor 30 located just proximal to distal tip electrode 18. Coronary sinus lead 16 is provided with a motion sensor 32 located just proximal to distal tip electrode 22. In the embodiment shown in FIG. 2, motion sensors 30 and 32 are embodied as receiver coils that are responsive to an (E)M field generated by excitation signal generator 40. Motion sensors 30 and 32 are each electrically coupled to sensing circuitry included in pacemaker 10 via insulated conductors extending from the distal to proximal ends of respective leads 14 and 16 and suitably connected to pacemaker 10 in a manner known in the art.

Endocardial lead 14 is shown with fixation members 34 used for anchoring the distal lead end at a desired right ventricular location such that tip electrode 18 and motion sensor 30 are positioned in a stable manner relative to the right ventricular heart wall. Inclusion of a distal fixation mechanism in a lead provided with a distal motion sensor may reduce extraneous lead movement as a source of error in wall motion measurements. It is assumed that lead 14 and lead 16 can be positioned relative to the heart such that motion of the distal lead ends imparted on motion sensors 30 and 32 is substantially due to heart wall motion. While two leads 14 and 16 are shown each having one motion sensor in the example embodiment of FIG. 2, the number of leads included in a system enabled for monitoring heart wall motion and the number of heart wall motion sensors employed may vary between embodiments. Furthermore, the types of leads used for carrying a motion sensor may vary and can include epicardial electrodes as well as the endocardial and coronary sinus leads shown in FIG. 2.

In operation, pacemaker 10 obtains data via electrodes and/or sensors deployed on leads 14, 15 and 16. This data is provided to processor 54, which suitably analyzes the data, stores appropriate data in memory 56, and/or provides a response or report as appropriate. In various embodiments, pacemaker 10 may activate an alert, select or adjust a therapy, and coordinate the delivery of the therapy by pacemaker 10 or another appropriate device.

Pacemaker 10 is equipped with telemetry circuitry and a telemetry antenna 65 for establishing a bidirectional communication link 47 with an external telemetry module 46 with external antenna 48. Data obtained or stored by pacemaker 10 can be uplinked to external telemetry module 46 via communication link 47. Likewise, programming data or interrogation commands can be downlinked from telemetry module 46 to pacemaker 10 via communication link 47.

Telemetry module 46 is typically incorporated in an external programmer or home monitor having a user interface to allow a clinician or other qualified personnel or a patient to enter commands or requests for transmission to pacemaker 10. An external programmer typically includes a display or other output module for presenting or storing data received from pacemaker 10. External programmers used in conjunction with programmable implantable medical devices are known in the art. A home monitor can be coupled to a communications network to allow transfer of data received from pacemaker 10 to a networked computer, central database, expert patient management center or other designated recipient or location. Wall motion data received by a home monitor and transferred for review by a clinician can be used for determining when adjustments of pacing parameters may be needed.

An external excitation signal generator 40 is provided for transmitting an excitation signal in a direction corresponding generally to the patient's heart, including the area in which a motion sensor 30 and/or 32 is positioned. In one embodiment, excitation signal generator 40 is provided as a (E)M field source for transmitting (E)M fields through the patient 6 so as to induce a current in motion sensors 30 and 32. Depending on the relative locations of sensors 30 and 32 and the direction of the transmitted (E)M field, a current signal may be induced in one or both of sensors 30 and 32. In embodiments including multiple lead-mounted motion sensors, induced current signals from multiple motion sensors may be obtained concurrently or sequentially.

In some embodiments, excitation signal generator 40 may alternatively transmit two or three (E)M fields in orthogonal directions to allow heart wall motion to be measured in two or three dimensions. In the case of two- or three-dimensional measurements, each of the (E)M fields are applied at a slightly different frequency such that the subsequently induced signals obtained from motion sensor 30 or 32 can be distinguished. Generally, orthogonally applied signals can be distinguished by adjusting some characteristic of the signals such as the frequency, phase, or time of the signals.

In an alternative embodiment, the motion sensors 30 and 32 and signal source 40 may be embodied as generally disclosed in the catheter mapping system and method described in U.S. Pat. No. 5,983,126 issued to Wittkampf, incorporated herein by reference in its entirety. Signal source 40 is provided as a low power RF source for transmitting an excitation signal, applied in one or more dimensions, through the patient using electrodes attached to the patient. The motion sensors 30 and 32 are provided as electrodes for measuring the time-varying voltage signal induced by the RF source. Relative changes in wall motion may be determined from the time-varying signal in one or more dimensions. Alternatively, the system may include a reference electrode as disclosed in the Wittkampf patent for use in determining an absolute position of the motion sensor electrode 30 or 32. By determining the absolute position frequently during a cardiac cycle, wall motion measurements can be computed.

The motion sensor signal can be processed to determine various signal parameters such as peak-to-peak difference of the motion signal over a cardiac cycle or the maximum first derivative of the motion signal during a cardiac cycle as a measurement of wall motion. These signal parameters, which can be determined under different physiological, pharmaceutical, hemodynamic, or pacing conditions, can be used to estimate relative changes in wall motion, such as relative changes in the total excursion of the endocardial wall over a cardiac cycle or in the peak endocardial acceleration.

When a three orthogonal (E)M fields are applied, the absolute location of the motion sensor 30 or 32 can be determined. By computing the absolute location of motion sensor 30 or 32 frequently during a cardiac cycle, absolute changes in position of the motion sensor 30 or 32 over a cardiac cycle can be used for deriving a wall motion measurement.

Computation of the location of motion sensor 30 or 32 during application of three orthogonally applied (E)M fields may be performed according to the image-guided surgical navigation methods implemented in the StealthStation™ surgical navigation system, manufactured by Medtronic, Inc. Reference is made, for example, to the surgical navigation methods generally described in U.S. Pat. No. 6,491,699 issued to Henderson, et al., incorporated herein by reference in its entirety.

The wall motion measurements determined by processor 44 are provided to output module 42 for presentation to a user. Output module 42 may be provided as a display and may include an electronic storage medium for storing wall motion data. Output module 42 may include a communication network interface for transmitting wall motion data to a clinician, central database, or expert patient management center.

The external components 40, 42, 44, and 46 shown in FIG. 1 used for generating an excitation signal, receiving motion sensor signals from an implantable device, computing motion sensor location and presenting wall motion data may be incorporated in a single external device, such as an external programmer, or implemented in more than one external device. For example telemetry module 46 and excitation signal generator 40 may be incorporated in an external programmer interfaced with a computer including processor 44 and output module 42.

Generally, relative differences in wall motion will be sufficient to detect changes in cardiac function in response to changes in programmed pacing parameters or other conditions. In some embodiments, however, the wall motion sensor signal may be calibrated such that wall motion parameters derived from the sensor signal may be computed in physical units. To enable calibration of the motion sensor signal, a second motion sensor is positioned on the same lead at a known distance from the first motion sensor. The known distance between the two motion sensors can be used for calibration purposes, for example as generally disclosed in the above-referenced Wittkampf patent.

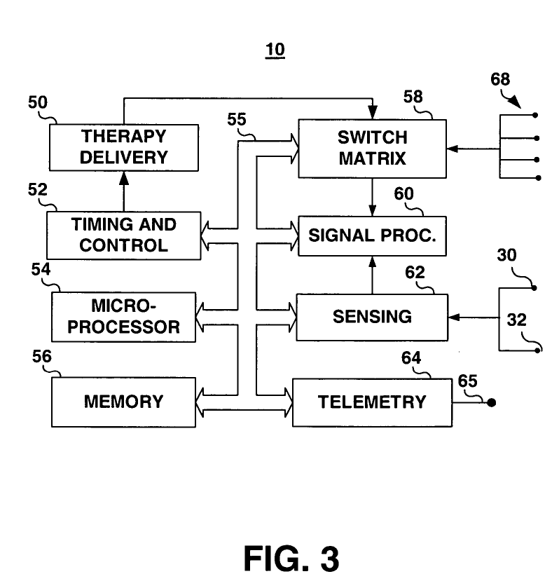

FIG. 3 is a block diagram of typical functional components included in an implantable cardiac stimulation device, such as pacemaker 10 shown in FIG. 1. Pacemaker 10 generally includes timing and control circuitry 52 and an operating system that may employ microprocessor 54 or a digital state machine for timing sensing and therapy delivery functions in accordance with a programmed operating mode.

Microprocessor 54 and associated memory 56 are coupled to the various components of pacemaker 10 via a data/address bus 55. Pacemaker 10 may include therapy delivery unit 50 for delivering a pacing therapy under the control of timing and control 52. Therapy delivery unit 50 is typically coupled to two or more electrodes 68 via a switch matrix 58. Switch matrix 58 is used for selecting which electrodes and corresponding polarities are used for delivering electrical stimulation pulses.

Each of the various modules shown may be implemented with computer-executable instructions stored in memory 56 and executing on processor 54, or in any other manner. The exemplary modules and blocks shown in FIG. 3 are intended to illustrate one logical model for implementing a pacemaker 10 having wall motion monitoring capabilities, and should not be construed as limiting. Indeed, the various practical embodiments may have widely varying software modules, data structures, applications, processes and the like. As such, the various functions of each module may in practice be combined, distributed or otherwise organized in any fashion in or across a medical device system that includes physiological signal sources.

Electrodes 68 may also be used for sensing electrical signals within the body, such as cardiac signals, or for measuring impedance. Cardiac electrical signals are sensed for determining when an electrical stimulation therapy is needed and in controlling the timing of stimulation pulses. Electrodes used for sensing and electrodes used for stimulation may be selected via switch matrix 58. When used for sensing, electrodes 68 are coupled to signal processing circuitry 60 via switch matrix 58. Signal processor 60 includes sense amplifiers and may include other signal conditioning circuitry and an analog to digital converter. Electrical signals may then be used by microprocessor 54 for detecting physiological events, such as detecting and discriminating cardiac arrhythmias.

Pacemaker 10 is coupled to one or more motion sensors 30 and 32. Motion sensor signals are received by sensing circuitry 62. Sensing circuitry 62 includes amplifiers and filters for receiving induced signals from motion sensors 30 and 32 and removing motion artifact such as motion due to respiration or body movement. Motion sensor signals may be received by signal processing module 60 for analog-to-digital conversion or other signal processing steps. Microprocessor 54 receives the motion signals and may perform computations for determining heart wall motion measurements. Microprocessor 54 provides motion signal data to telemetry module 64 for uplink to an external telemetry module 46 (shown in FIG. 2).

Pacemaker 10 may additionally or alternatively be coupled to various physiological sensors used for monitoring a patient or detecting the need for a cardiac stimulation therapy. Such sensors may include pressure sensors, flow sensors, blood chemistry sensors, activity sensors or other physiological sensors known for use with implantable medical devices.

The operating system includes associated memory 56 for storing a variety of programmed-in operating mode and parameter values that are used by microprocessor 54. The memory 56 may also be used for storing data compiled from sensed physiological signals, such as wall motion signals, and/or relating to device operating history for telemetry out on receipt of a retrieval or interrogation instruction. All of these functions and operations are known in the art, and many are generally employed to store operating commands and data for controlling device operation and for later retrieval to diagnose device function or patient condition.

Pacemaker 10 further includes telemetry circuitry 64 and antenna 65. Programming commands or data are transmitted during uplink or downlink telemetry between pacemaker telemetry circuitry 64 and external telemetry circuitry included in a programmer or monitoring unit as described previously. Telemetry circuitry 64 and antenna 65 may correspond to telemetry systems known in the art.

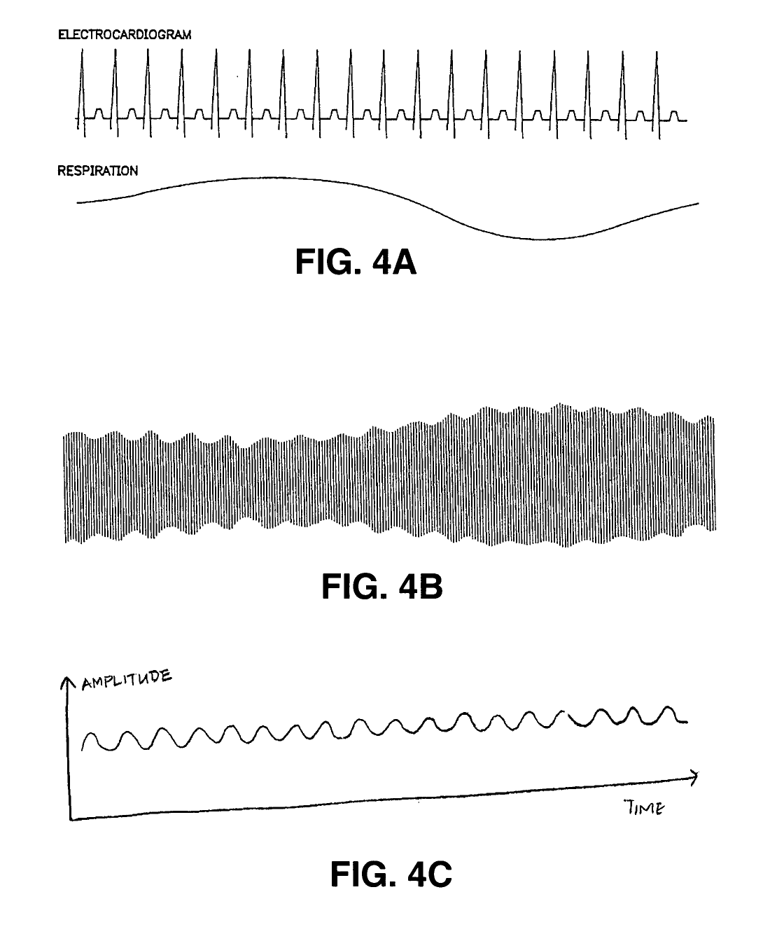

FIG. 4A shows an electrocardiogram and a respiration signal. FIG. 4B shows a signal sensed from a motion sensor. The sensed motion sensor signal is sampled at a frequency high enough to contain cardiac wall motion information. The amplitude of the motion sensor signal is shown to vary due to respiration and myocardial contraction. The respiration motion artifact can be removed using a bandpass filter, resulting in a cardiac wall motion signal as shown in FIG. 4C. The cardiac wall motion signal of FIG. 4C can be can be used to compute a signal characteristic that can be used as a surrogate measure of average peak wall excursion, peak endocardial acceleration, or other wall motion parameter useful in assessing heart function. Relative comparisons of the computed signal characteristic under different conditions, for example under different pacing parameter settings, allows for an assessment of myocardial function under the different conditions.

In some embodiments, the electrocardiogram signal shown in FIG. 4A or an internally obtained cardiac EGM signal is used to sample the cardiac wall motion signal in a time-gated manner. For example, R-wave sensing may cause the sensing circuitry to sample the cardiac wall motion signal at a desired frequency for a predetermined sensing window. Alternatively, the wall motion signal may be sampled at a predetermined interval following a sensed R-wave for a desired number of cardiac cycles. A series of time-gated wall motion signal samples or other time-averaging techniques can be used to determine a characteristic wall motion parameter.

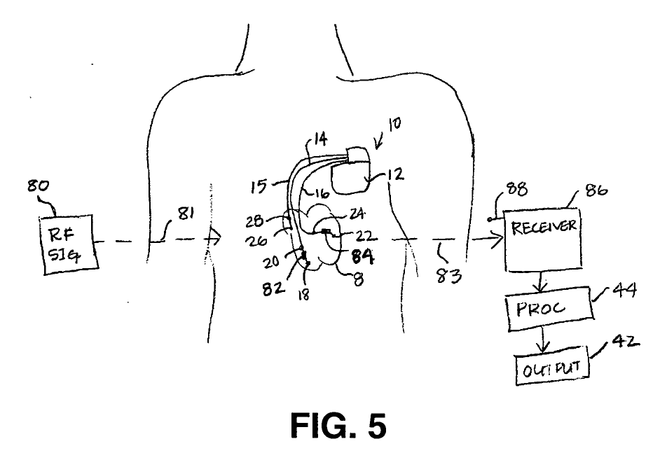

FIG. 5 is a schematic diagram of a wall motion monitoring system according to an alternative embodiment of the present invention. Motion sensors 82 and 84 carried by right ventricular endocardial lead 14 and coronary sinus lead 16, respectively, are embodied as electronic “tags” in the form of an RF resonant circuit. The excitation signal generator is embodied as an RF signal generator 80, which produces an electronic field 81 that matches the resonant frequency of motion sensors 82 or 84. In systems including a single motion sensor, RF signal generator 80 produces an electronic field matching the resonant frequency of the single motion sensor. In systems including multiple motion sensors, RF signal generator 80 may produce an electronic field having multiple frequencies for matching unique resonant frequencies for each individual motion sensor. The excitation signal generator 80 and electronic “tag” motion sensors 82 and 84 can be embodied in a manner similar to systems used for electronic article surveillance systems. Reference is made, for example, to U.S. Pat. No. 5,825,291 issued to Platt et al., and to U.S. Pat. No. 6,836,216 issued to Manov et al., U.S. Pat. No. 5,121,103 issued to Minasy et al., all of which patents are incorporated herein by reference in their entirety.

Sensing circuitry is provided as a receiver 86 with antenna 88 for collecting the resonance signal 83 produced by sensor 80 or 82 at a frequency high enough to reliably track motion sensor movement over a cardiac cycle. In systems including multiple motion sensors, resonance signal 83 may contain multiple resonance frequencies associated with each unique sensor such that the location of multiple sensors may be tracked simultaneously over a cardiac cycle. Receiver 86 provides the collected resonance signal to processor 44 for computation of wall motion measurements provided as output 42, for display, transmission or storage.

In various embodiments of the invention, different types of excitation signals and motion sensors may be used based on RF, EM, acoustical, or other forms of energy or hybridized versions of these types of signals. Generally an excitation signal is generated to induce a lead-mounted motion sensor signal that becomes time-varying due to cardiac wall motion.

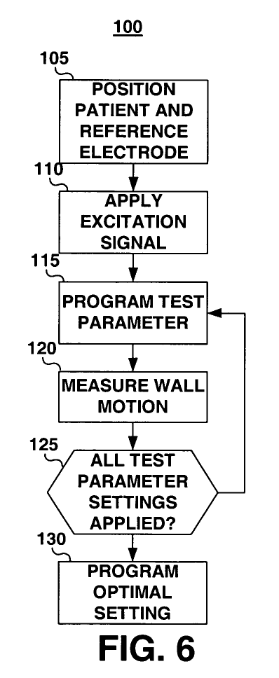

FIG. 6 is a flow chart summarizing steps included in a method for optimizing pacing parameters using a wall motion signal in accordance with the invention. At step 110, the external (E)M field is applied to the patient, which may include one, two, or three orthogonally arranged signals for measuring a wall motion signal in one, two or three dimensions respectively.

At step 115, a pacing parameter is programmed to a selected test setting. The cardiac response to the programmed setting is measured by measuring the wall motion at step 120 by collecting the induced (E)M signal in the motion sensor. A characteristic parameter of the induced signal may be determined. The process of measuring wall motion using the motion sensor can be repeated for any number of desired test parameter settings. For example, for optimization of dual chamber pacing, wall motion measurements may be performed for a number of AV interval settings. During cardiac resynchronization therapy, wall motion measurements may be performed for a number of VV and/or AV interval settings. After testing all desired test parameter settings, as determined at decision step 125, the optimal setting based on wall motion measurements can be programmed. Generally, the optimal setting corresponds to the setting at which the wall motion parameter being measured is maximized (i.e., optimal contractility) at step 130. In some applications, however, an optimal setting may correspond to a setting resulting in a wall motion parameter that is less than the maximum value measured.

Thus a cardiac lead with a motion sensor and an associated system and methods for use have been described. It is recognized that one having skill in the art and the benefit of the teachings provided herein may conceive of numerous variations to the embodiments presented herein. The systems and methods described are intended to be illustrative embodiments of the invention and should not be construed as limiting with regard to the following claims.

What is claimed is:

1. A system, comprising:
a cardiac lead having a motion sensor disposed at or near the distal lead end;
an external excitation signal source;
a sensing module for receiving a signal from the motion sensor produced in response to the external excitation signal source; and
a processor for computing a measurement of cardiac wall motion using the motion sensor signal received from the sensing module.

2. The system of claim 1, wherein the sensing module is a receiver coil.

3. The system of claim 2, wherein the external excitation signal source is an electromagnetic field generator.

4. The system of claim 3, wherein a plurality of orthogonal electromagnetic fields are generated from the electromagnetic field generator.

5. The system of claim 1, wherein the sensing module is an electrode.

6. The system of claim 5, wherein the external excitation signal source is an RF signal generator.

7. The system of claim 1, wherein the sensing module is an electronic circuit that resonates in response to the signal.

8. A system, comprising:
means for generating an external excitation signal;
means for generating a time-varying signal in response to the excitation signal that varies in time due to cardiac wall motion;
means for receiving the time-varying signal;
means for computing a wall motion measurement using the time-varying signal.

9. The system of claim 8, wherein the means for generating the external excitation signal include an electromagnetic field generator.

10. The system of claim 9, wherein the electromagnetic field generator produces a plurality of electromagnetic fields in orthogonal orientation.

11. The system of claim 8, the means for generating the external excitation signal include an RF signal generator.

12. The system of claim 8, wherein the means for receiving include a receiver coil.

13. The system of claim 8, wherein the means for receiving include an electrode.

14. The system of claim 8, wherein the means for receiving include a resonant electronic circuit.

15. A method, comprising:
generating an external excitation signal;
receiving an induced signal from a wall motion sensor responsive to the external excitation signal, the induced signal being a time-varying signal that varies in time due to cardiac motion;
computing a wall motion measurement from the received signal.

16. The method of claim 4 further comprising adjusting a pacing parameter corresponding to a maximum computed wall motion measurement.

17. The method of claim 15, wherein generating the external excitation signal includes generating a first electromagnetic field.

18. The method of claim 18, wherein generating the external excitation signal further comprises generating a second electromagnetic field orthogonal to the first electromagnetic field.

19. The method of claim 15, wherein generating the external excitation signal includes generating an RF signal.
